(19) United States
(12) Patent Application Publication
(10) Pub. No.: US 2019/0365415 A1
(43) Pub. Date: Dec. 5, 2019

(54) NOVEL EQUIPMENTS AND METHODS FOR TRANSORAL ENDOSCOPIC SURGERY OF UPPER GASTROINTESTINAL TRACT USING RIGID PLATFORM

(71) Applicant: Sanjiv P. Haribhakti, Ahmedabad (IN)

(72) Inventor: Sanjiv P. Haribhakti, Ahmedabad (IN)

(21) Appl. No.: 16/475,649

(22) PCT Filed: Mar. 29, 2017
(86) PCT No.: PCT/IN17/50115

§ 371 Date: Jul. 2, 2019

(30) Foreign Application Priority Data
Jan. 3, 2017 (IN) 201721000186

(51) Int. Cl. A61B 17/34; A61B 1/273; A61B 1/00; A61B 1/05; A61B 1/07; A61B 18/14; A61L 29/02

(52) U.S. Cl. CPC A61B 17/3423; A61B 1/273; A61B 1/00135; A61B 1/00154; A61B 2017/00199; A61B 1/07; A61B 18/1485; A61L 29/02; A61B 2017/345; A61B 1/05

(57) ABSTRACT

The present invention relates to novel equipment's and methods for minimally invasive surgery emphasizing use of rigid platform for transoral endoscopic surgery of upper gastrointestinal tract. The present novel equipments and methods using the rigid platform aids in effective surgical procedures through natural orifice with accuracy, minimum bleeding, and under vision scarless major surgery of upper gastrointestinal (UGI) tract such as transoral cardiomytomy for achalasia cardia (TOEM), transoral fundoplication (TOF) for hiatus hernia, transoral bariatric surgery (TOBS), transoral oesophagectomy (TOO) for oesophageal cancers and NOTES UGI surgery. Present invention provides a Transoral Port (TOP) used for performing natural orifice surgery of the upper gastrointestinal tract; which mainly comprises of Sheath (SH), Blunt Introducer (BI), Telescope (TE), Diaphragm (DI), Silicon washers (SW), Tunnel pusher tube (TP), Airport (AP), Suction port (SP), Bevel (AB or PB), Markings (MA), Monitor (MO).

### FIELD OF THE INVENTION

The present invention generally relates to novel equipments and methods for minimally invasive surgery emphasizing use of rigid platform for transoral endoscopic surgery of upper gastrointestinal tract. The approach described here, of using transoral rigid platform for surgery of the esophagus, GE junction and stomach is absolutely novel.

The present invention aids in effective minimal access surgical procedures through natural orifice with high accuracy, excellent vision, minimum bleeding, and under vision scarless major surgery of upper gastrointestinal (UGI) tract such as cardiomyotomy for achalasia cardia, fundoplication for hiatus hernia, bariatric surgery for morbid obesity, oesophagectomy for oesophageal cancers, and pure NOTES UGI surgery.

### BACKGROUND AND THE PRIOR ART

The Upper gastrointestinal (UGI) tract involves the esophagus, stomach and proximal part of duodenum is a very important initial part of the digestive system, which starts in the neck and ends in the abdomen. The esophagus (foodpipe) propels the food from the mouth into the stomach for digestion. Major part of the esophagus is situated deep in the thorax (chest) and is surrounded by vital organs such as heart, lungs, aorta, azygous vein, trachea and bronchus. The stomach is a very important initial organ for digestion which breaks down food by the strong acid secreted in the stomach and stores the food for gradual emptying into the intestine for complete digestion. The stomach is a distensile organ, situated in the left upper abdomen, surrounded by vital organs such as spleen, left liver and pancreas, and is hidden deep under the left costal margin.

The access to UGI tract is difficult due to its location, and as such can be accessed traditionally by open or laparoscopic surgery which is also difficult due to its deep location and being surrounded by vital organs.

The diseases of UGI tract such as gastro-esophageal reflux disease (GERD) are very common. Currently lifestyle disorders such as GERD and Obesity are significantly on the rise world over. Other common problems are achalasia cardia and cancer of the esophagus. The major concern in UGI diseases is its deep location, difficult access and that they are surrounded by vital organs.

The major concerns in UGI tract/disease conditions/include achalasia cardia, fundoplication for hiatus hernia, bariatric surgery for morbid obesity, esophagectomy for esophageal cancers,

Gastro-esophageal reflux (GERD) is a common clinical problem affecting patients at all ages but more so in the prime of their active life. Some of these patients have significant reflux or complications and require surgery for hiatus hernia to improve their quality of life and prevent complications.

Achalasia cardia is a condition of hypertrophy of lower esophagus leading to difficulty in swallowing (dysphagia). The only effective treatment for these patients is cardiomyotomy ie releasing these hypertrophic muscles in the lower esophagus to relieve their symptoms of dysphagia and avoiding the complications such as aspiration pneumonitis. Currently cardiomyotomy is being done with the laparoscopic technique. However recently peroral endoscopic myotomy (POEM) has also being performed.

Morbid Obesity is an endemic and a serious lifestyle disorder the world over. Laparoscopic bariatric surgery currently is the gold standard and is considered the only effective long term treatment for morbid obesity. Most of these surgeries are performed with laparoscopic technique currently. The gold standard operations are Laparoscopic Gastric Sleeve and Laparoscopic Gastric Bypass. Recently few of these procedures have been performed with an endoscopic approach called endoluminal bariatric procedures.

Cancer of the Esophagus is a serious illness as it causes dypshagia ie difficulty in swallowing. Esophagectomy ie removal of the tumour with esophagus, the only curative option for these patients with cancer of the esophagus is one of the most formidable operations of mankind with a high morbidity and a significant risk of mortality. Currently these procedures are performed by open or thoraco-laparoscopic techniques.

For diagnosis of such disease conditions of UGI tract and to understand its severity, for planning the surgeries, endoscopes were invented. Recent diagnostic approaches involve flexible endoscopy. Before flexible endoscopy was invented in the early 1980's, most of the diagnosis of UGI tract i.e. diseases of esophagus was done by a rigid instrument called Rigid Esophagoscope. This was rigid tube with a cold light source and a channel for taking biopsy. This rigid tube was mainly used for diagnosis of upper GI conditions and for some minor procedures such as biopsy and foreign body removal. There was no video technology available at that point of time and no surgical procedures were possible with this basic rigid esophagoscope. After introduction of flexible endoscopy, these rigid esophagoscopes soon became obsolete.

Flexible endoscopy such as Gastroscopy and Colonoscopy have been used for diagnosis and treatment of various GI conditions since the early 1980's. In the last 4 decades, flexible endoscopy has rapidly developed and is used extensively in gastroenterology for diagnosis as well as treatment of several gastrointestinal diseases.

Further developments lead to some more advanced procedures done through the flexible platform such as POEM (Peroral endoscopic myotomy) done for a condition called achalasia cardia. Though flexible endoscopy is very safe for diagnosis, it has several limitations when it comes to advanced treatment of gastrointestinal diseases. Thus, said endoscopes were limited to diagnosis enabling planning of surgeries.

Post 1990's has been an era of minimally invasive surgery. Since the first introduction of Laparoscopic Cholecystectomy in 1989, most of the abdominal surgeries are performed through multiple small keyholes termed as Laparoscopic surgery. These minimally invasive surgery have an advantage that they are highly patient friendly, and that patient recovers much faster as they have much less pain after surgery, less bleeding during surgery, and they have a cosmetically better outcome. These surgeries require the use of telescope connected to video camera and multiple Laparoscopic instruments which are long and narrow instruments with different configurations used to perform these surgeries. However, these laparoscopic surgery needs multiple small openings on the abdomen and multiple scars are left behind permanently.

Since 2000, a new method of minimal access surgery was developed called SILS (Single incision laparoscopic surgery). In SILS, from a single incision, multiple 2-4 instruments are inserted into the abdomen to perform the surgery. By placing this single incision into the umbilicus, the scars can be hidden and cosmetically this can be more appealing to patients. However, this kind of SILS surgery are more difficult for surgeons, as there is no triangulation and there is crowding and fighting of instruments. To overcome these limitations, several new curved instruments for SILS surgery have also been developed by instrument manufacturing companies. Even in SILS, a single scar is permanently left behind and risk of wound and scar related problems persist.

More recently, in the last decade, a new approach of surgery called NOTES (Natural orifice transluminal endoscopic surgery) is being developed, whereby surgery is done through the natural orifice such as anal canal called Transanal surgery. Various NOTES approaches described from transgastric or transrectal access over the flexible platforms were developed. NOTES Surgery with flexible platform did not evolve over the last decade due to several technological limitations and violation of basic surgical principles.

More recently since 2010, flexible endoscopy has been used for performing Peroral endoscopic myotomy (POEM) for treatment of achalasia cardia. The standard method for this procedure is Laparoscopic cardiomyotomy. Flexible Endoscopy has also been used to treat selected patients with gasto-esophageal reflux disease (GERD) and bariatric (weight loss) procedures.

### Disadvantages of the Prior Art

There exists various surgical equipments and methods for performing surgery of UGI tract. However, they suffer at least one of the following disadvantages:
1. Flexible endoscopic platform fails to effectively assist in the surgery of GI tract when advanced surgical procedures are required to be performed. This is because of following reasons:
   i. Coaxial vision and instrumentation—In flexible endoscopy, the instrument channel is in the flexible body of the endoscope. Thus when the instrument movements are done to perform surgery, the vision also moves, thus failing to qualify for a steady surgical platform. This is one of the reasons why natural orifice trans luminal surgery (NOTES) did not develop on the flexible endoscopy platform in the last ten years inspite of lot of enthusiasm for NOTES surgery in early 2000 after performance of transluminal cholecystectomy and appendectomy from stomach and colon in animals.
   ii. Instrument channel—On the flexible endoscopy platform, there is only one instrument channel as a standard which severely limits performance of major surgical procedures. Special endoscopes were also developed by a few companies with two surgical instrument channel but these could not be successfully used in humans for performance of NOTES. These prototypes were not standardized in humans and had significant limitations in terms of user interface and performance of surgical procedures by endoscopists.
   iii. Suturing—Any surgical procedure requires dissection, cutting and stitching or suturing the opened part. Endoscopic flexible platform has an inherent limitation as suturing is not possible of opened mucosa as required in POEM. Thus endoscopists use clips to close the mucosal defect. In other situations where suture repair is needed as in Fundoplication for hiatus hernia, endoscopy uses a very special device which is prohibitively expensive for suturing called transoral incisionless fundoplication (TIF) by Esophyx™ device. This is so expensive that it is rarely used in western and developed countries and is unheard of in developing countries. The main reasons for the high cost are complexity of construction, and high skill needed.
   iv. Haemostasis—In any surgical procedure there is always a risk of bleeding. Adequate control of bleeding is one of the most important part of the surgery. This becomes even more important in minimal access surgery where blood reflects light in a small space and obscures the vision. Thus control of bleeding becomes even more paramount and important in minimal access surgery. The NOTES approach involves working in very small areas and thus it is important to control any bleeding while doing surgery. In flexible platforms methods used to control bleeding involves monopolar probe coagulation (gold probe) and use of clips. However, it does not facilitate the use of bipolar forceps that can be used to control bleeding. Flexible platforms also fail to facilitate packing of the area that can be performed for brisk bleed and suture control of bleed, unlike rigid platforms for surgery.
   v. Complications—Surgical procedures are bound to develop some complication in a small number of patients, in spite of all precautions. Any endoscopic procedure may develop post-operation complications which rarely may require re-intervention. Flexible platforms fail to facilitate the management of such complications.
   vi. Air insufflation—Air insufflation routinely used in diagnostic flexible endoscopy is not advisable in surgeries. It has been found that prolonged air insufflations can rarely lead to air embolism which can be a fatal complication. Thus in advance endoscopic surgery, carbon dioxide (CO2) insufflations is used instead of air insufflations for distension of operative field to perform the surgery. CO2 is also rapidly absorbed by the intestine and is highly soluble in blood, and is rapidly excreted through lungs and kidneys. In any case, measurement of ETCO2 is necessary during the surgery.
2. Use of conventional rigid oesophagoscopy imparted following disadvantages and thus failed to effectively assist in surgery of UGI tract through transoral route:
   i. Chances of injury—The prior arts with rigid surgical equipments had chances of injury to the patient's oropharyngeal area or to upper aero-digestive tract, while inserting the surgical equipment through the patient's transoral route.
   ii. Fixity—there exists problems pertaining to fixity of the rigid platform after its introduction into the Esophagus, wherein the platform needs to be fixed so that, with undue or unnecessary movements, the platform does not get removed inadvertently. However, the conventional rigid platforms failed to provide any provision facilitating its fixation in said region.
   iii. Instrument clashing—One of the major limitation of existing platforms is it fails to facilitate the use of multiple surgical instrument for surgery due to constructional defects, leading to instrument clashing in turn leading to difficulties in managing such surgeries.
   iv. Air leakage—there exists a problem of air leakage (CO₂) leading to insufficient CO₂ insufflation; which is necessary to distend the organ where surgery is to be performed. Thus, it fails to distend the area of surgical work directly.
   v. Suction—One of the major issues in this surgery is fogging due to the generation of smoke during electrocoagulation. This results in difficulties in providing clear vision for surgery. This is also a demerit of flexible endoscopy used for surgeries because withdrawal of telescope requires withdrawal of theendoscope. The smoke needs to be rapidly evacuated from the surgical field for providing clear vision. However, conventional platforms of said surgery fails to provide so.
   vi. End on view—Because of a long straight channel and straight trajectory of the conventional instruments, it becomes difficult to perform the surgery in the wall of the esophagus. Thus, such methods failed to motivate the surgeons to use them for said surgery.
   vii. The rigid diagnostic esophagoscope used only one instrument channel, thus major surgery or suturing is not possible on the traditional rigid esophagoscope.
3. The Standard Laparoscopic surgery used today for these cases has the limitation that multiple small openings need to be created on the abdomen to perform these operations. These wounds may create pain or wound complications such as wound infection or wound hernias. These leave behind multiple scars on the abdomen which may be cosmetically not very appealing.
Thus, there is an unmet need to develop novel equipments and methods for transoral endoscopic surgery of upper gastrointestinal tract (Esophagus, GE junction and Stomach) (TOES-UGI) using rigid platform through natural orifice with accuracy, minimum bleeding, and under vision scarless major surgery of upper gastrointestinal (UGI) tract such as transoral cardiomyotomy (TOEM) for achalasia cardia, transoral fundoplication (TOF) for hiatus hernia, transoral bariatric surgery (TOBS) and transoral natural orifice (NOTES) oesophagectomy for oesophageal cancers that obviates the problems of the prior art.

### OBJECTS OF THE INVENTION

Principal object of the present invention is to create novel equipments and methods for transoral endoscopic surgery of upper gastrointestinal tract (Esophagus, GE junction and Stomach) (TOES-UGI) using rigid platformthrough natural orifice with accuracy, minimum bleeding, and under vision scarless major surgery of upper gastrointestinal (UGI) tract such as transoral cardiomyotomy (TOEM) for achalasia cardia, transoral fundoplication (TOF) for hiatus hernia, transoral bariatric surgery (TOBS) and transoral natural orifice (NOTES) esophagectomy for esophageal cancers that obviates the problems of the prior art.

Yet another object of the present novel equipments and methods for transoral endoscopic surgery of upper gastrointestinal tract using rigid platform is to provide a novel Transoral port (TOP) also be called Operating Esophagoscope for Esophageal (TOP-E) and Gastric surgeries (TOP-G).

Yet another object of the present novel equipments and methods for transoral endoscopic surgery of upper gastrointestinal tract using rigid platform is to develop novel instrumentations for transoral surgery and SILS and NOTES platform which can be used from a single incision laparoscopic surgery (SILS) platform and particularly can be used for long and narrow single incision ports such as transoral ports.

Yet another object of present novel equipments and methods for transoral endoscopic surgery of upper gastrointestinal tract using rigid platformis to develop new scarless approaches to existing surgeries such as Transoral approach to endoscopic myotomy (TOEM) for Achalasia Cardia, Transoral Fundoplication (TOF) for Gastro-esophageal reflux disease (GERD), Transoral approach for bariatric surgery (TOBS), Transoral approach for esophagectomy for esophageal cancers (TOO) and Pure NOTES UGI surgery.

Yet another object of present novel equipments and methods for transoral endoscopic surgery of upper gastrointestinal tract using rigid platform is to perform scarless and painless major surgery of the upper GI tract by avoiding incisions and thereby to eliminate wound related complications unlike conventional laparoscopic surgery.

Yet another object of present novel equipments and methods for transoral endoscopic surgery of upper gastrointestinal tract using rigid platform is to provide cosmetically superior transoral surgery obviating the problems of conventional laparoscopic surgery.

Yet another object of present novel equipments and methods for transoral endoscopic surgery of upper gastrointestinal tract using rigid platform is to overcome the limitations of Flexible endoscopy by performing two handed instrument dissection, performance of suturing and improving the safety and efficacy of the procedure as it is performed by the surgeon on a rigid platform which is a standard for minimally invasive surgery.

Yet another object of present novel equipments and methods for transoral endoscopic surgery of upper gastrointestinal tract using rigid platform is to eliminate the chances of injury by providing a blunt introducer (BI) that enables insertion of the present transoral port (TOP) into the transoral route of the patient without causing injury to its oropharyngeal area or to upper aero-digestive tract. Moreover, said blunt introducer is provided with under vision with a 5 mm telescope to ensure the same.

Yet another object of present novel equipments and methods for transoral endoscopic surgery of upper gastrointestinal tract using rigid platform is to obviate the problems of fixity of esophagoscope.

Yet another object of present novel equipments and methods for transoral endoscopic surgery of upper gastrointestinal tract using rigid platform is to eliminate the instrument clashing by providing specially designed curved instruments and related techniques.

Yet another object of present novel equipments and methods for transoral endoscopic surgery of upper gastrointestinal tract using rigid platform is to facilitate the surgical procedure in the wall of the esophagus or stomach.

Yet another object of present novel equipments and methods for transoral endoscopic surgery of upper gastrointestinal tract using rigid platform is to develop surgical equipment that enables development of a sub-mucosal tunnel.

Yet another object of the present novel equipments and methods for transoral endoscopic surgery of upper gastrointestinal tract using rigid platform is to develop novel approaches for natural orifice transluminal surgery (NOTES) of gastrointestinal tract.

### BRIEF DESCRIPTION OF DRAWINGS

FIG. 1A: Shows the schematic view of present novel equipment for transoral endoscopic surgery of upper gastrointestinal tract using rigid platform while surgeon uses Transoral Port (TOP) for said surgery.

FIG. 1B: Shows the enlarged schematic view of present novel equipment for transoral endoscopic surgery of upper gastrointestinal tract using rigid platform while patient is operated using Transoral Port (TOP) for said surgery.

FIGS. 1C1 & 1C2: Shows Esophageal transoral port (TOP-E) of the present novel equipment for transoral endoscopic surgery of upper gastrointestinal tract using rigid platform for doing Esophageal surgeries with posterior bevel and anterior marking.

FIG. 1D: Shows Gastro transoral port (TOP-G) of the present novel equipment for transoral endoscopic surgery of upper gastrointestinal tract using rigid platform for doing surgeries in Gastric or stomach region (below diaphragm) with anterior bevel and anterior markings.

FIG. 1E: Shows top view of silicon washer of the first embodiment TOP.

FIG. 1F: Shows top view of silicon washer of the second embodiment TOP.

FIG. 1G: Shows top view of silicon washer of the third embodiment.

FIG. 1H: Shows top view of silicon washer of the tunnel pusher sheath.

FIG. 1I: Shows the top view and side view of tunnel pusher sheath.

FIG. 1J: Tunnel pusher sheath with bevel.

FIG. 1K: Blunt introducer for TP sheath.

FIG. 1L: Tunnel pusher sheath assembly.

FIG. 1M: Shows fragmented view of the present TOP.

FIG. 2: Shows various instruments used in present novel equipments for transoral endoscopic surgery of upper gastrointestinal tract using rigid platform.

FIG. 2A: L hook with proximal straight tip and curved distal end.

FIG. 2B: L hook with proximal curved tip and curved distal end.

FIG. 2C: Shows blunt or atraumatic grasper.

FIG. 2D: Shows star electrode.

FIG. 2E: Shows J tip needle.

FIG. 2F: Shows endoscopic view of transoral surgery.

FIG. 3: Shows various applications of present novel equipment for transoral endoscopic surgery of upper gastrointestinal tract using rigid platform.

FIG. 3A: Shows Transoral endoscopic myotomy (TOEM) for achalasia cardia.

FIG. 3B 1: Shows TOP-G with knot pusher for TOF.

FIG. 3B2: Shows completed TOF with 3 sutures at 9'oclock, 12'o clock and 3'o clock position.

FIG. 3B3: Shows Endoscopic view of FIG. 3B2.

FIG. 3C: Shows Transoral Gastric sleeve (TOGS) as part of Transoral bariatric surgery (TOBS).

FIG. 3D: Shows Transoral endoscopic Esophagectomy (TOO).

### MEANING OF REFERENCE NUMERALS OF SAID COMPONENT PARTS OF PRESENT NOVEL EQUIPMENTS AND METHODS FOR TRANSORAL ENDOSCOPIC SURGERY OF UPPER GASTROINTESTINAL TRACT USING RIGID PLATFORM

| | |
|---|---|
| P | Present novel equipment for transoral endoscopic surgery |
| | of upper gastrointestinal tract using rigid platform |
| SH | Sheath |
| BI | blunt Introducer - Under Vision |
| DI | Diaphragm |
| SW | Silicone washer |
| TE | Telescope |
| IN | Instruments for Transoral surgery |
| AP | Air port |
| SP | Suction port |
| MO | Monitor |
| AB | Anterior Bevel |
| PB | Posterior Bevel |
| KP | knot Pusher |
| TP | Tunnel pusher |

### DETAILED DESCRIPTION OF THE INVENTION

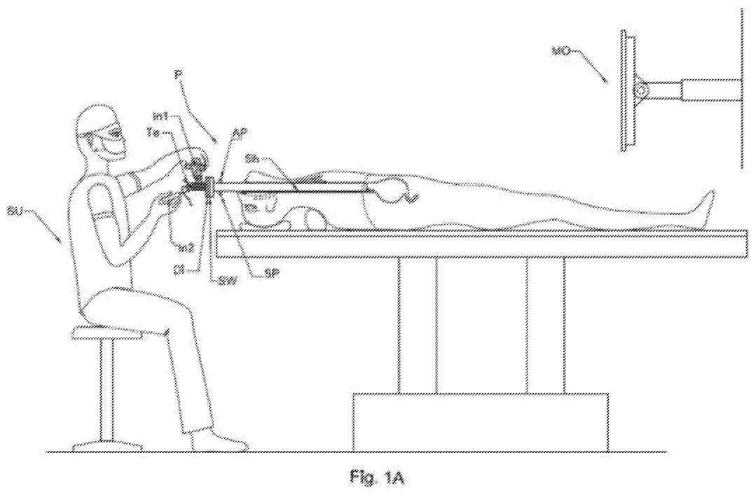

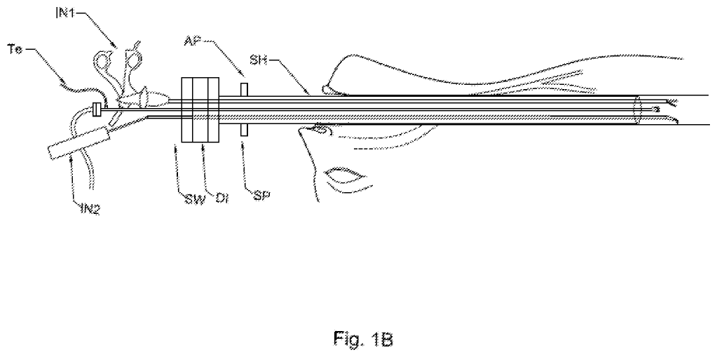

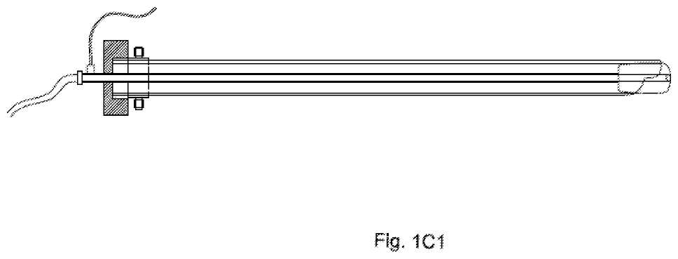

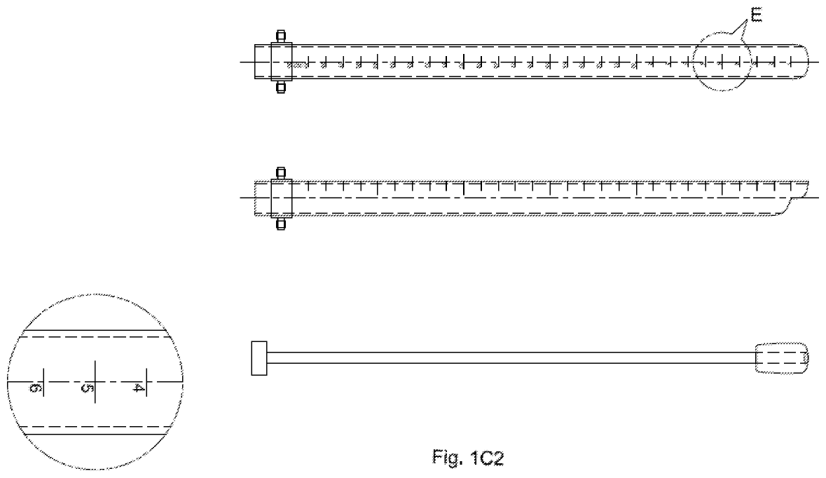

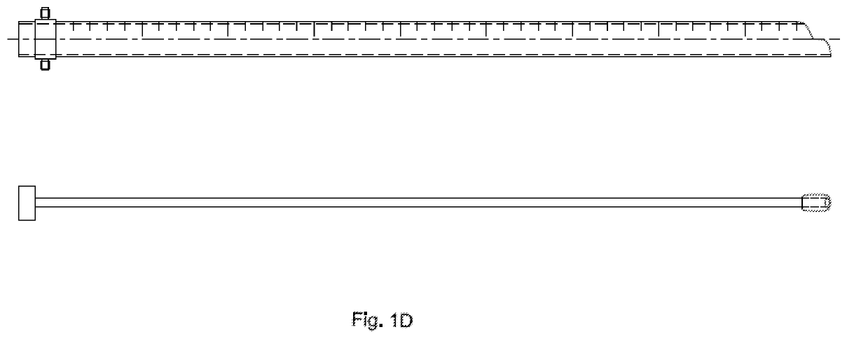

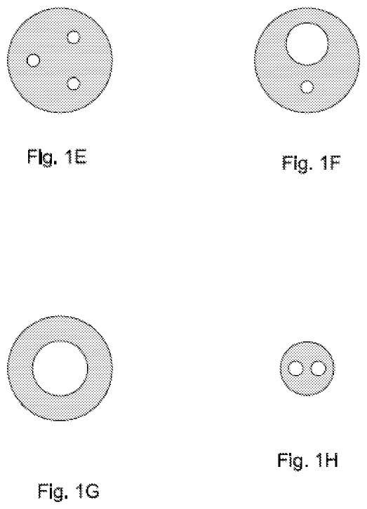

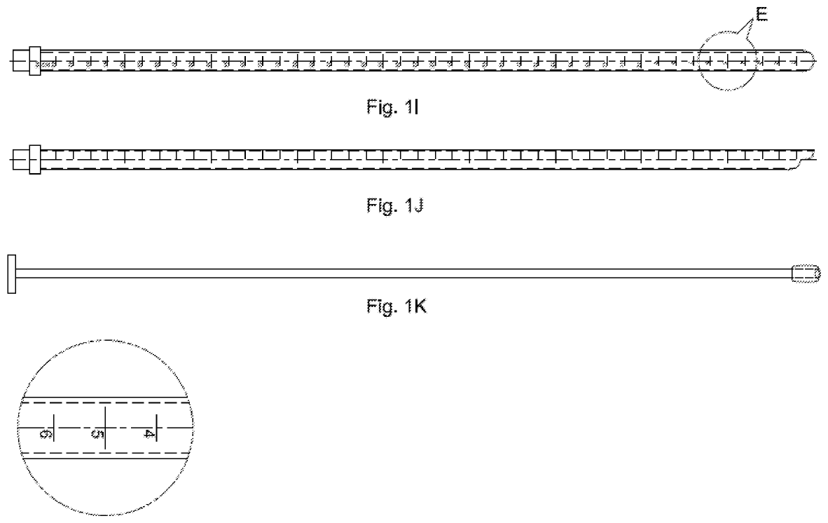

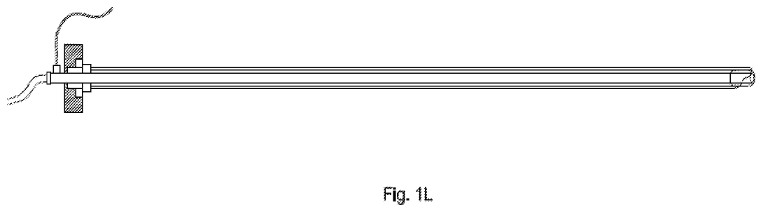

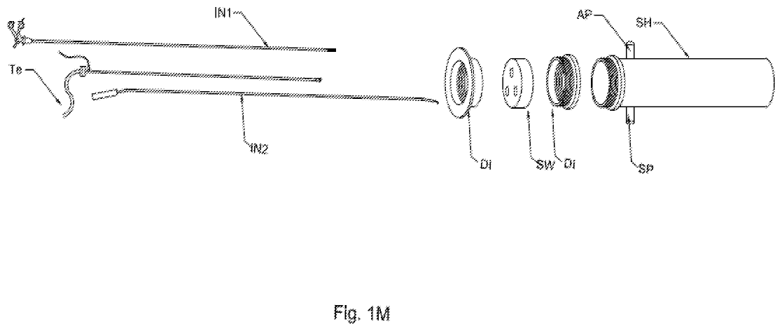

The present novel equipments and methods for transoral endoscopic surgery of upper gastrointestinal tract using rigid platform (P) embodies, in its main embodiment; a novel Transoral port (TOP) also be called Operating Esophagoscope for Esophageal (TOP-E) and Gastric surgeries (TOP-G). (Refer FIG. 1C and FIG. 1D) Said Transoral Port (TOP) is made from rigid material such as SS 316 or Titanium so that it can be biocompatible, sterilizable by autoclaving and reusable. The present novel equipments and methods using the rigid platform aids in effective surgical procedures through natural orifice with accuracy, minimum bleeding, and under vision scarless major surgery of upper gastrointestinal (UGI) tract such as transoral cardiomytomy for achalasia cardia (TOEM), transoral fundoplication (TOF) for hiatus hernia, transoral bariatric surgery (TOBS), transoral esophagectomy (TOO) for esophageal cancers and NOTES UGI surgery that obviates the problems of the prior art.

### The Main Embodiment

Referring to FIG. 1A to 1H and FIG. 1M, the main embodiment of the present invention is the Transoral Port (TOP) used for performing natural orifice surgery of the upper gastrointestinal tract. As they are rigid ports, they are made of SS or Titanium to make it light weight, and easily autoclavable and biocompatible. The main embodiment embodies two types of Transoral Ports (TOP) based on its applicability:
i. Transoral Port for surgery of the Esophagus (TOP-E) which is having a working length of 30 cm and is specially designed for performing cardiomyotomy for achalasia cardia (TOEM) and NOTES Esophagectomy. (FIG. 1C)
ii. Transoral port for surgery of the GE junction and Stomach (TOP-G) which is having a working length of 45 cm, which is specially designed to perform Transoral Fundoplication (TOF), Transoral bariatric surgery (TOBS) and NOTES UGI surgery. (FIG. 1D)

Each of these ports have a 20 mm internal working diameter. They are constructionally same; except for lengths.

Said Transoral port (TOP) of main embodiment of the present novel equipments for transoral endoscopic surgery of upper gastrointestinal tract using rigid platform (P) mainly comprises of following parts:
Sheath (SH),
Blunt Introducer (BI),
Telescope (TE),
Diaphragm (DI),
Silicon washers (SW),
Tunnel pusher tube (TP),
Air port (AP),
Suction port (SP),
Bevel (AB or PB),
Markings (MA),
Monitor (MO);

### (Refer FIG. 1M)

wherein:

Said Sheath (SH) is a hollow tube made from Stainless Steel. It is inserted from access route or the natural orifice; including transoral route and it remains in the patient through which the instruments pass into the body to perform surgery. Said sheath (SH) is introduced with the help of blunt introducer (BI) which has an incorporated 5 mm channel to introduce telescope and insert the Transoral Port (TOP) under vision. Said sheath (SH) has insufflations (AP) and suction ports (SP) which are also used as fixation device. Based on the application, the Transoral Port (TOP) has varied lengths. Preferred embodiments include:
1. Sheath (SH) for transoral port of esophagus (TOP-E) with diameter of 20 mm (from 15 to 25 mm), and a working length of 30 cm (from 25 to 40 cm) for Esophageal surgery. Said sheath (SH) passes from the mouth through the upper oesophageal (cricopharyngeal sphincter) into the esophagus under direct vision with the help of blunt introducer (BI).
2. Sheath (SH) for Transoral surgery for GE junction & Gastric surgery (TOP-G) such as Fundoplication and bariatric surgeries has a length of 45 cm (varying from 40 to 60 cm with a diameter of 2 cm. Said sheath (SH) passes from the mouth through the upper oesophageal (cricopharyngeal sphincter) into the stomach under direct vision with the help of blunt introducer (BI).

Said Diaphragm (DI) is made from biocompatible Teflon and Stainless Steel (SS) so as to not to make the entire port very heavy. It is screwed air tight to the sheath (SH) on which a silicon washer is applied. The diaphragm (DI) is constructed in two parts in between which the silicon washer (SW) is applied. The construction is such that the silicon washer remains fixed to the diaphragm, allows smooth passage of instruments, and prevents gas leak and that the silicon washer can be easily changed during the surgery if needed.

Said Silicon Washers (SW) fit in between the diaphragm (DI) and are made of biocompatible Silicon and have plurality of openings with airtight seal to insert multiple long and narrow (laparoscopic) instruments (IN1, IN2) through it into the sheath (SH). Said airtight seal prevents gas leakage. Said washer (SW) can be changed for different steps of the surgery depending on the nature of instrument to be used. Different embodiments for present Transoral Port (TOP) include:
1. A washer (SW) for 20 mm sheath (SH) has three openings of 5 mm diameter each in a first embodiment of the present invention (P). Said washer can be designated as 5,5,5 washer. Said three openings are utilized to insert into the sheath (SH), while doing the surgery, a 5 mm telescope and two 5 mm instruments (IN1, IN2). (FIG. 1E)
2. A washer (SW) for 20 mm sheath (SH) has two openings of 5 mm and 10 mm diameter each in a first embodiment of the present invention (P). Said washer can be designated as 10,5 washer wherein a 5 mm telescope and one 10 mm instrument are inserted through the openings. (FIG. 1F)
3. A washer (SW) for 20 mm sheath (SH) has one opening of 12 mm diameter each in a first embodiment of the present invention (P). Said washer can be designated as 12 washer, and is used to insert the tunnel pusher (TP) sheath through the TOP. (FIG. 1G)

Said Telescope (TE), in a preferred embodiment is a high definition (HD) telescope to provide a clear image, preferably it is a 5 mm telescope of around 50 cm (varying from 30 cm to 60 cm) length with video camera attached on it; and is used to provide the vision for surgery. On the telescope, a fibre-optic light cable is attached for providing light for the surgery. The telescope is generally held by the assistant surgeon.

Said Air port (AP) is used for CO₂ insufflation at a flow of 10-20 L/min with a pressure of 15 mm Hg (varying from 10-20 mm Hg). This distends the esophagus or stomach and creates a space for surgery.

Said Suction port (SP) is used for suction of smoke while using the electrocautery during the surgical procedures.

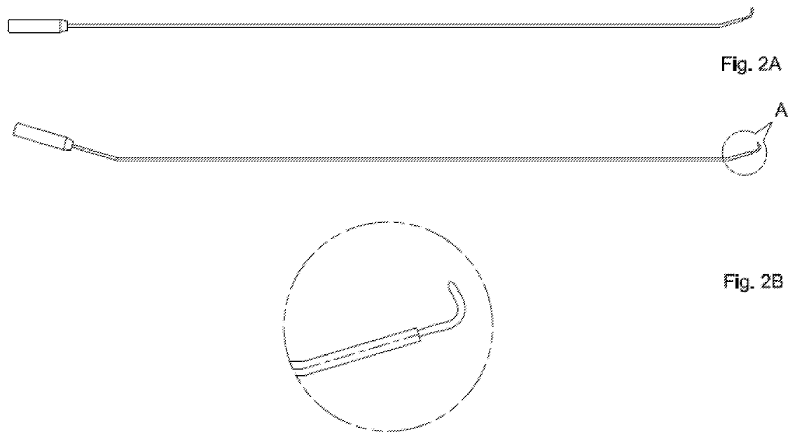

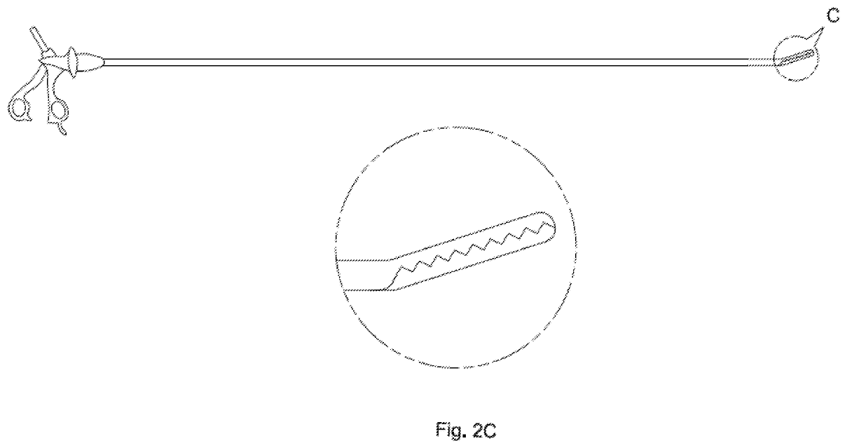

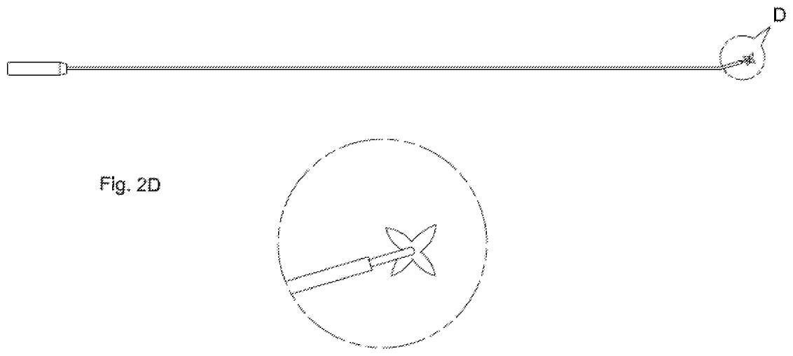

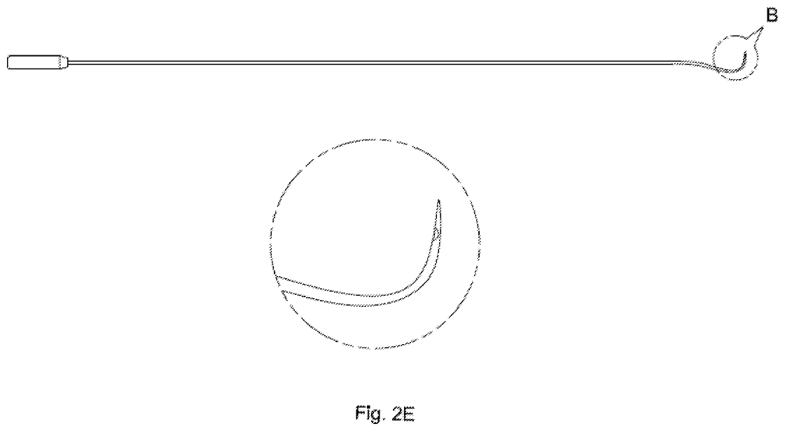

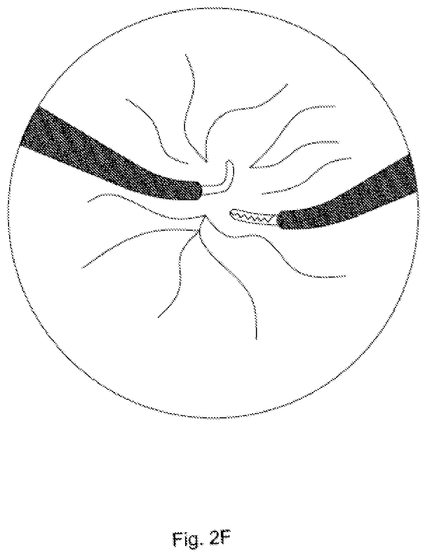

Said Instruments (IN1, IN2) Plurality of long preferably 35 cm to 50 cm depending on sheath (SH) used (varying from 20 cm to 60 cm), and thin preferably 2 to 5 mm (varying from 1 mm to 6 mm) instruments are used of different configurations for performing the surgical procedure through present rigid platform. Said instruments include maryland dissector, atraumatic graspers, hook electrode, star electrode, needle holder, suction, bipolar cautery etc. The present invention (P) also embodies special instruments that are developed having a curved tip for working in present Transoral Port (TOP) with small diameter. They also have curves on the proximal end to avoid instrument fighting. These instruments are either 3/5 mm and have an extra length of 50 cm. Said special instruments include:
Star Electrode It is designed for creating submucosal space and performing myotomy in TOEM. Said star electrode is a three point star electrode with an active tip. (FIG. 2D).
J tip needle along with thread retriever is developed to facilitate suturing of horizontal defects in a small space. (FIG. 2E).

Said Sheath (SH) has bevel at its distal end which is a cut out to perform surgery wherein:
1. Said sheath (SH) of the Transoral Port (TOP) for Esophagus (TOP-E) has a bevel at its distal end which is a cut out to engage the esophagus posteriorly for performing posterior cardiomyotomy. The bevel is for 10 mm depth (varying from 5 to 20 mm) and occupying 50% circumference (ranging from 25-75% circurmference). (FIG. 1D)
2. Said Sheath (SH) of the Transoral Port (TOP) for GE junction and Gastric Surgery (TOP-G) has a cutout (bevel) anteriorly on its distal end where fundoplication needs to be performed. The bevel or cutout is anteriorly to perform an anterior Dorr fundoplication. (FIG. 1C)
3. Said Sheath (SH) of the Transoral Port (TOP) for Esophagus (TOP-E) and Stomach (TOP-G) have markings on their anterior surface for visibility and orientations for the surgeon, as to the surface of the organ, whether he/she is anterior or posterior and as to the position of the TOP to the length of esophagus or stomach. (FIG. 1C to FIG. 1I)
4. Said Tunnel Pusher sheath (TP) is made of Stainless Steel (SS) and is 12 mm diameter (varying from 8 to 16 mm) and has a length of 45 cm (varying from 40 to 60 cm). It has two ports at proximal end for insufflation and smoke evacuation. The tunnel pusher sheath (TP) is also inserted under vision with a blunt introducer (BI). Said Tunnel Pusher silicon washer is 5,5 washer to accommodate one 5 mm telescope and another ⅗ mm instrument. (FIG. 1I, FIG. 1J, FIG. 1K)

### Present Novel Methods for Transoral Endoscopic Surgery of Upper Gastrointestinal Tract Using Rigid Platform

Referring to FIG. 1A, 1B:
1. The patient is placed on the operation table under general anesthesia in an extended neck position for passing and keeping present novel equipments for transoral endoscopic surgery of upper gastrointestinal tract using rigid platform (P) from the mouth into the esophagus; when the transoral surgery is to be performed. The sandbag under the shoulder extends the neck naturally and facilitates the passage of the rigid port and helps to keep the port in this position during the surgery. The patients head is supported on a ring. The Surgeon sits on a chair or stool facing the patient at a comfortable height. The assistant surgeon is to the left of the surgeon and assistant to the right of the surgeon. The monitor (MO) provides a clear image of the surgical field to the surgeon and the assistant surgeon. It is preferably HD monitor of large size 26 or 32 inches.
2. Said Sheath (SH) is inserted under vision with a blunt introducer (BI) inserted into the mouth. The blunt introducer (BI) facilitates introduction of said sheath (SH) into the body through natural orifice without injury to adjoining structures. In the centre of the blunt introducer (BI) is a tunnel to admit a 5 mm 0 degree telescope so that the entire introduction from mouth through the throat and into the esophagus is done under direct vision of the endoscope preferably with some insuffflation of CO₂ through said Air Port (AP) on said sheath (SH). The insertion is facilitated by using a direct laryngoscope to retract the tongue and avoid any injury to the oropharyngeal structures.
3. Once the port is inserted to the desired length into the esophagus, the blunt introducer (BI) and the telescope (TE) is removed, the sheath (SH) of present transoral port (TOP) is fixed with fixation sutures to air port (AP) and the suction ports (SP) to the face of the patient. Additional fixation of the said the sheath (SH) of present transoral port (TOP) can be done with tapes with the face of the patient or with the operating table. Once the Transoral Port (TOP) is passed under vision into the esophagus, the neck is flexed slightly to optimise the position.
4. At the proximal end of the sheath (SH), there is a screwing mechanism for an attachment of the diaphragm (DI).
5. The diaphragm (DI) has two metallic component inbetween which the silicon washer (SW) is applied, which enables to introduce multiple 3 mm or 5 mm laparosopic instruments through its plurality of openings.
6. With the introduction of telescope and one or two instruments the surgery can be started for any application.
   Applications of Present Novel Equipments and Methods for Transoral Endoscopic Surgery of Upper Gastrointestinal Tract Using Rigid Platform and Comparison of Applications with the Prior Art

The applications of the present invention (P) describes herein after includes the applications of Transoral Port (TOP) for surgery of esophageous (TOP-E) and for stomach (TOP-G) respectively.

Applications of Esophageal Transoral Port (TOP-E):

Referring to FIG. 1C, Transoral Port (TOP-E) is used as novel equipments and methods for transoral endoscopic surgery of upper gastrointestinal tract using rigid platform (P) for following applications for the surgery in esophagus:

### 1. Transoral Endoscopic Myotomy (TOEM) for Achalasia Cardia

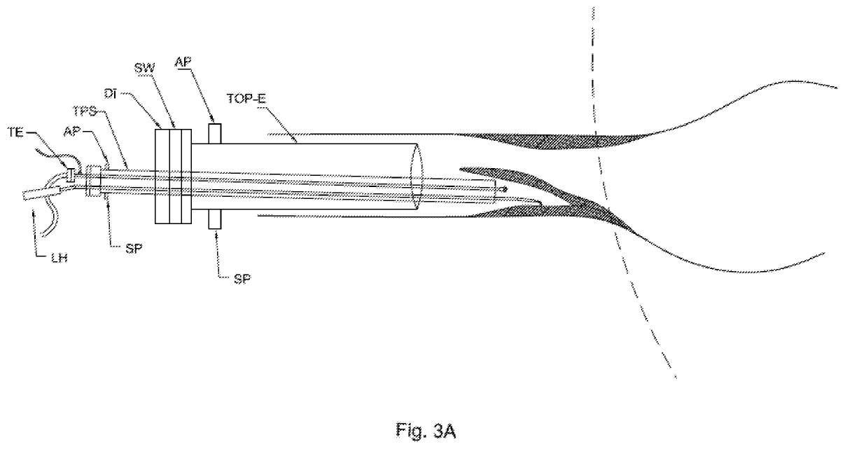

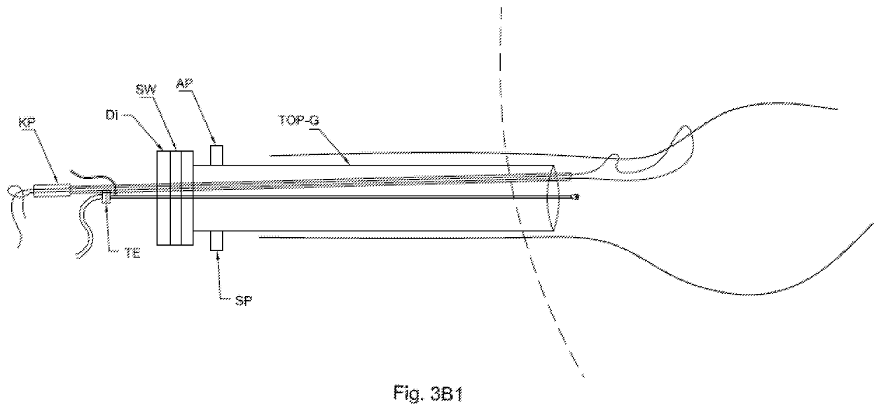

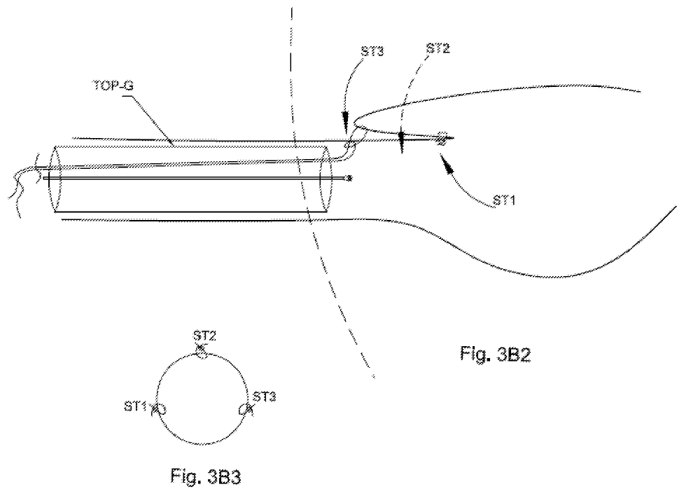

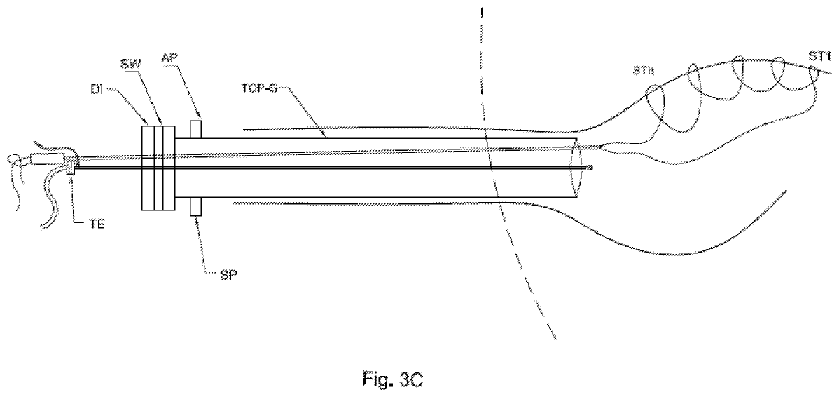

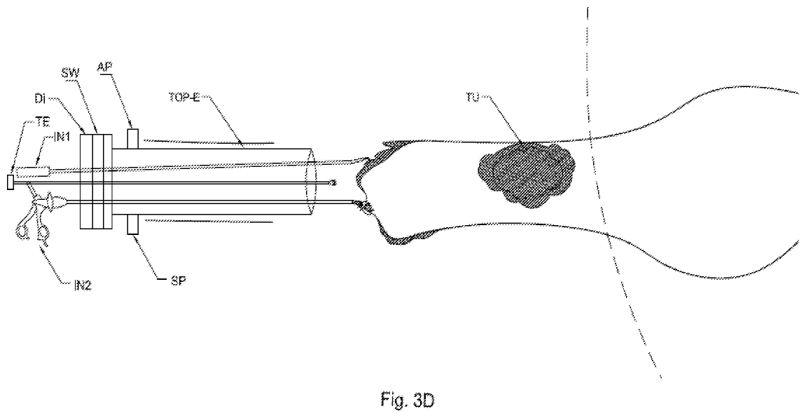

The existing methods for treatment for Achalasia Cardia is called Laparoscopic cardiomyotomy (Prior Art). The present invention (P) performs the said surgery of cardiomyotomy with transoral approach. This procedure is being termed as transoral endoscopic myotomy (TOEM) (Refer FIG. 3A). Present transoral equipments and methods for transoral endoscopic surgery of upper gastrointestinal tract using rigid platform (P) imparts following advantages over the prior used technique:

| | Laparoscopic | Transoral |
|---|---|---|
| | cardiomytomy | cardiomyotomy |
| | LCM | TOEM |
| Access | Abdominal | Oral |
| Incsion, Wounds | Multiple | None |
| Scars | Multiple | None |
| Pain | Yes, Mild | None |
| Recovery | Fast | Faster |
| Complications | Low | Lower |

Recently cardiomyotomy has also been performed with flexible endoscopy called peroral endoscopic cardiomyotomy (POEM). The comparison with prior art POEM is described here:

| | Peroral | |
|---|---|---|
| | endoscopic | Transoral |
| | myotomy (POEM) | endoscopic |
| | (Prior Art) | myotomy (TOEM) |
| Platform | Flexible endoscopy | Rigid transoral |
| | | platform |
| Vision & | Coaxial | Independent |
| Instruments | | |
| Instrument | Single | Dual |
| channels | | |
| Personnel | Endoscopist | Surgeon |
| Procedure | Since last 5-7 | Just Innovated |
| | years | |
| Mucosal closure | Clips | Sutures |
| Funodplication | Not possible | Possible |

### Present Novel Method for TOEM Includes Following Steps:

Once the Transoral Port (TOP) is inserted under vision, said Blunt Introducer (BI) is removed, diaphragm (DI) is applied and the platform is fixed to the patient with two sutures. The sheath (SH) is fixed in such a way that the bevel (PB) is posterior and the markings (MA) are anterior. A diagnostic flexible endoscopy is then performed through the transoral port to assess the distance of the GE junction and plan the myotomy and the tunnel length. Insufflation with CO₂ is started and ETCO2 monitoring is done by the anesthetist to ensure that hypercarbia does not occur. Painting of local oesophageal mucosa is done with dilute Povidone Iodine and Flushing with Saline is done. The procedure of endoscopic cardiomyotomy is now started. Following steps follows:

A 5 mm Telescope (TE) 30 degree angled, 50 cm long telescope is chosen to perform the myotomy. This telescope (TE) is used with standard laparoscopic instruments to perform the myotomy. Alternatively a standard 5 mm 30 degree telescope (TE) is used with long bariatric length instruments to perform the myotomy. Using differential length of instruments and telescope (TE) is to avoid instrument and telescope (TE) clashing in a very small space of 2 cm diameter of present transoral port (TOP).

The first step in this surgery is to create a submucosal space in the esophagus. Injection of dilute 1:100 methylene blue is done by an aspiration needle in the submucosal space to create a mucosal bulge. This lifts the mucosa and allows a submucosal tunnel to be created. 10-20 cc of dilute methylene blue is injected and mucosal bulging is observed.

A mucosal incision is placed starting at 6' o clock position on the esophagus to perform a posterior myotomy. A longitudinal incision is preferred so that mucosal closure with suturing becomes easier in view of the horizontal suture line, which is then closed with the J tip needle of the special instruments (IN1 or IN2) of the present invention (P). Alternatively a vertical mucosal incision is used and closed with standard suturing done with needle holder or endostitch (IN1, IN2). Another alternative is to do an anterior myotomy which has shown to have equivalent results as posterior myotomy.

The next step is to create sub-mucosal tunnel. Gentle dissection by a maryland dissector creates a space in the submucosal area for accommodating a balloon. A CRE balloon from 10-12 mm is used to create a submucosal space so that the submucosal pusher tube is inserted into the newly created submucosal space.

A Tunnel pusher (TP) Tube of 12 mm diameter and a 5,5 silicon washer (SW) along with blunt under vision introducer (BI) with CO2 insufflation is inserted into the submucosal space that has been created. Insufflation with CO₂ is started to enlarge this space. Under vision of a 5 mm 0 degree telescope (TE) and a hook or a triangular star instrument (IN1, IN2) which is slightly curved at the tip, the submucosal space is developed distally into the esophagus. A long 12-15 cm of submucosal tunnel is created, comprising approx. 10-12 cm in the esophagus and around 2-3 cm into the stomach. The tunnel is elongated by going beyond GE junction into stomach for 2-3 cm. The crisscross fibres of the GE junction are apparent and signifies the presence of GE junction. Small submucosal blood vessels are carefully coagulated and divided to prevent bleeding. If bleeding is encountered, control is achieved with bipolar coagulation forceps. Care is taken to do each step under direct vision and avoiding mucosal perforation.

Once the tunnel is complete, the myotomy is started—For myotomy, a tunnel pusher (TP) tube of 45 cm length with a posterior bevel (PB) is used to perform a posterior myotomy. A 5 mm 30 degree angled telescope (TE) is preferred to perform the myotomy. The myotomy is restricted to the circular fibres or alternatively a full thickness myotomy is performed. The recommended length of myotomy is approx. 10-12 cm out of which, approx 8-10 cm is in esophagus and 2-3 cm into stomach. The myotomy is started about 2-3 cm. distal to the mucosal opening into the esophagus to create a flap like effect upon closing the mucosa. Once the myotomy is complete beyond the GE junction, haemostasis is achieved. The myotomy is performed by a ⅗ mm hook electrode or a star electrode with a cutting or a blended current. It is advisable that all steps are done under vision. One special problem of smoke evacuation needs to be addressed due to the narrow diameter of the platform. Several options to circumvent this problem is by using a hook with inbuilt suction port (SP), simultaneous cautery and suction machine, foot controlled suction, dual suction etc. To evacuate the smoke, the telescope may need to be withdrawn repeatedly while cautery is being used.

The mucosal closure is performed with sutures or endostitch. For a suture closure of horizontal incision, said special instrument (IN1/IN2) i.e. J tip needle device is used to pass the needle with thread which is then retrieved with a special retriever instrument (IN1/IN2) which are 2 mm in diameter. Alternatively, for closing a vertical incision, a vicryl 3/0 is used on a 27 mm ⅝th circle needle. The needle is grasped by the needle holder and taken into the esophagus from the Transoral Port (TOP). The stitch is taken without dislodging the needle. The needle is grasped and extracted from the Transoral Port (TOP). An extracorporeal knot is preferred and tightened. Another simple or figure of 8 suture is taken and this completes the mucosal closure. Generally 2-3 sutures are necessary to close the mucosal incision. For endostitch closure, a new Silicon washer 10,5 is needed to be applied on the Diaphragm (DI) of the Transoral Port (TOP). A 10 mm endostitch is passed into the esophagus with an angled 5 mm telescope of 30 degree. Endostitch is taken on both edges of the mucosa and endostitch is removed and an extracorporeal knot is tied and pushed into the esophagus with a knot pusher (KP).

### 2. Transoral Endoscopic Esophagectomy (TOE) or Hybrid NOTES Esophagectomy (HNE) (Refer FIG. 3D)

Current surgical treatment for cancer of the esophagus involves Esophagectomy and Gastric replacement done either with open or Thoraco-Laparoscopic approach.

| | | NOTES |
|---|---|---|
| | Open Esophagectomy | Esophagectomy |
| | Standard of Care | Just Innovated |
| Approach | Open Surgery | Transoral, Neck, |
| Umbilcus | | SILS |
| Invasion | Highly invasive | Minimally invasive |
| approach | approach | |
| Pain | Very severe & | Minimal & Short |
| | Prolonged | lived |
| Bleeding | Much more | Minimal |
| ICU Stay | Prolonged | Short |
| Recovery | Delayed | Early |

Endoscopic approach to cancer esophagus involves palliation for advanced cancer such as use of self expandable stents, laser and other ablative therapies. There are no reports of endoscopic therapy or transoral approach to surgical resection of resectable cancer of the esophagus.

Present Transoral port (TOP-E) has shown various above mentioned advantages in said treatment. Said Transoral Port (TOP) is used to divide the esophagus proximal to the tumour and come out into the mediastinum, just as is done in transanal TME for rectal cancer. Under vision, entire mediastinal esophagectomy and periesophageal lymphadenectomy is performed without disturbing the mediastinal pleura or the azygos vein. Neck incision is used to apply a SILS port for performing the mediastinal dissection of the esophagus. The gastric tube mobilization and specimen retrieval is performed from periumbilical NOTES approach and SILS surgery from umbilicus. The Hammock retractor (Authors innovation) (Fig. ?) is used for atraumatic and scarless retraction of the left lobe of the liver during this surgery. Finally neck anastomosis between the pulled up gastric tube and the cervical esophagus is fashioned. Alternatively a stapled anastomosis is created in the neck.

### Present Novel Methods for Transoral Endoscopic Oesophagectomy (TOO):

The TOP-E is inserted into the esophagus just proximal to the tumour and fixed in position. Insufflation of CO2 is started.

A purse string suture is taken into the port and the esophagus is closed 1-2 cm proximal to the tumour. The knot is again tied extracorporeally and pushed into the esophagus.

An incision is placed encircling the esophagus and is deepened through the esophagus to enter into the mediastinum. The incision is extended all around in the esophagus. Care is taken not to injure mediastinal structures including trachea, bronchus and azygos veins. With the help of blunt grasper in one hand and hook or harmonic scalpel in another help the entire esophagus is completely separated from adjoining structures. Haemostasis is achieved. Peritoneum is entered and then SILS umbilical approach is used to complete the abdominal part of the procedure. A a complete NOTES esophagectomy is achieved by inserting the TOP-G port into the abdomen after peritoneal entry and performing the abdominal steps from the transoral route.

Applications of Gastric Transoral Port (TOP-G):

Referring to FIG. 1D, Transoral Port (TOP-G) is used as novel equipments and methods for transoral endoscopic surgery of upper gastrointestinal tract using rigid platform (P) for following novel NOTES applications for the surgery in Stomach:

### 3. Transoral Endoscopic Fundoplication (TOF) for Hiatus Hernia

Hiatus Hernia is currently treated by Laparoscopic Fundoplication, which involves 5 incisions on abdomen.

Transoral Endoscopic Fundoplication (TOF) is a novel approach of doing antireflux procedure from a transoral approach. For this, a specially created Transoral Port (TOP-G) (refer FIGS. 3B1 and 3B2) which is a long 45 cm platform with 2 cm diameter is inserted from the mouth into the esophagus and then a partial anterior Dorr Fundoplication is done by hitching the fundus of the stomach to the GE junction with three interrupted sutures to create an angulation of the GE junction and create an antireflux barrier to prevent gastro esophageal reflux. This operation is also done in conjunction with a cardiomyotomy to prevent reflux commonly seen after cardiomyotomy. This technique is similar to Laparoscopic anterior partial (Dorr) Fundoplication. This approach has the potential to treat many patients with gastro-esophageal reflux with minimal or a small hiatus hernia with a NOTES transoral approach. This technique is called Transoral Fundoplication (TOF).

| | Laparoscopic | Transoral |
|---|---|---|
| | Fundoplication-Prior Art | Fundoplication |
| | Standard of care | Just Innovated |
| Applicability | All cases of Severe GERD | Selected patients |
| Approach | Abdomen - Laparoscopic | NOTES - Transoral |
| Incisions | 5 incisions on abdomen, | Incisionless |
| | can be done SILS | |
| Results | Good | Better |

Recently endoscopic methods of fundoplication called transoral incisionless fundoplication (TIF) has been developed. Early results are encouraging for selected cases with mild reflux and small hiatal hernia. The comparison with the prior art is described here:

| | Transoral | |
|---|---|---|
| | incisonless | Transoral |
| | Fundoplication | Fundoplication |
| | (TIF) Prior Art | (TOF) |
| Platform | Flexible | Rigid |
| Personnel | Endoscopist | Surgeons |
| Equipment | Special, Expensive | No special |
| | | equipment |
| Suturing/Repair | Special equipment | Hand suturing/ |
| | | Endostitch |
| Under vision | Partially | Completely |
| Simulate | No | Yes |
| Fundoplication | | |

### Present Novel Method for TOF Includes Following Steps:

Alongwith TOEM, Said Transoral Endoscopic Fundoplication (TOF) is performed before doing a myotomy preferably, as it does not compromise the myotomy or the oesophageal mucosal closure while placing the transoral ports (TOP). Alternatively it is done after the myotomy.

The steps for Transoral Fundoplication (TOF) either for hiatus hernia or for achalasia cardia is the same and is described below:

Sheath (SH) of Transoral Port (TOP) for Gastric surgery (TOP-G) of 45 cm length, with 2 cm diameter is inserted under vision of a 5 mm telescope into the esophagus. The sheath (SH) has an anterior bevel to perform anterior Dorr fundoplication. A new Silicon washer 10,5 is attached to Diaphragm (DI) for suturing with endostitch 10 mm. The suturing of the fundus with the GE junction is performed with assistance of any of the following techniques:
1. Endoscopic guidance—a Screw like instrument (IN1/IN2) is inserted from the instrument channel of a flexible 10 mm endoscope (TE) to hook the fundus of stomach after retroflexion and pull the fundus; said endoscope (TE) is then taken out keeping the instrument channel in position; Endostitch is then inserted from the 10 mm port, suturing of the fundus with the anterior part of GE junction performed; extracorporeal knotting is preferred over intracorporeal knotting; wherein three such sutures are placed at 9, 12, 3 o'clock position (ST1, ST2, ST3). (refer FIG. 3B2, 3B3).
2. Laparoscopic guidance—A 5 mm telescope (TE) at umbilicus gives a vision of the GE junction with the help of Hammock retractor retracting the left lobe of the liver. A 3 mm trocar is placed just underneath the xiphoid process and a 3 mm blunt instrument depresses anterior fundus of the stomach. Under this vision, transoral suture is passed by the transoral surgeon. This approach needs a two team approach and two endovision systems simultaneously to perform the fundoplication. Similarly three sutures are taken with laparosopic assistance and all are tied. Alternatively from a SILS port at the umbilicus and three 5 mm ports and a Hammock retractor in position to retract the left lobe of the liver, an anterior Dorr fundoplication is performed from a completely laparoscopic U NOTES approach.
3. Flexible paediatric endoscopic guidance—A 5 mm, 100 cm long flexible paediatric endoscope is used to assist taking fundal sutures by using a rat tooth grasper from the instrument channel of 2.2 mm to pull the fundus and take the sutures hitching it to the GE junction.
4. J Needle—said J needle (IN1/IN2) is also be used to perform the fundoplication with along port with the help of the thread retriever.

### 4. Transoral Endoscopic Bariatric Surgery (TOBS)

Transoral endoscopic bariatric procedures include Transoral endoscopic gastric sleeve—TOGS, revisional procedures for weight gain after gastric bypass surgery such as Transoral revision after bariatric surgery—TORBS and transoral gastric bypass (TOGB). (refer FIG. 3C)

Most of these surgeries for Morbid Obesity are performed with laparoscopic technique currently. Common prior art techniques are Laparoscopic Gastric Sleeve and Laparoscopic Gastric Bypass. The comparison with the prior art ie laparoscopic bariatric surgery is mentioned herewith:

| | Laparoscopic Bariatric | Transoral Bariatric |
|---|---|---|
| | surgery | Surgery |
| | Current standard | Just innovated |
| Approach | Abdominal | Transoral |
| Incisions | 5 ports | Incisionless |
| Scars | Small scars on abdomen | No scars |
| Cosmesis | Moderate | Excellent |

Recently few of these procedures have been tried with an endoscopic approach called endoluminal bariatric procedures.

| | | Transoral |
|---|---|---|
| | Endoluminal bariatric | bariatric |
| Surgery | procedures | procedures |
| Introduction | Recent | Just Innovated |
| Platform | Flexible Endoscopy | Rigid Surgical |
| Personnel | Endoscopists | Surgeons |
| Equipments | Special, Expensive | Standard |
| | | Surgical |

The transoral approach is used for performing bariatric or weight loss surgeries notably gastric plication or Transoral Gastric sleeve (TOGS) which is currently one of the most common and effective bariatric procedures. Many patients regain weight after laparoscopic bariatric surgery. These patients need revisional bariatric surgeries. Recently many revisional procedures are also being done with endoluminal approach. Transoral rigid platform is used to perform revisional bariatric procedures such as revisional gastric sleeve or refashioning of anastomosis after Lap. Gastric bypass surgery. Transoral gastric Bypass (TOGB) is also a valid option for selected patients. The transoral approach is herald a new era in bariatric treatment called as Transoral Bariatric Surgery (TOBS). The benefit of these operations are completely incisionless, scarless and painless NOTES approach for treatment.

### Present Novel Methods for Transoral Bariatric Surgery (TOBS) Includes Following Steps:

Transoral gastric Sleeve (TOGS)—The first step is introduction of TOP-G into the stomach. The next goal is to plicate the greater curvature of the stomach with multiple sutures to reduce the capacity of the stomach. This is done at several levels of the stomach starting from distal stomach and gradually progressing towards the proximal stomach. The knots are tied extracorporeally and pushed back into the stomach. At the end of the procedure the capacity of the stomach is reduced. This is done with plication sutures or even stapling guns currently available introduced from the transoral approach.

For revision of laparoscopic gastric bypass, when the anastomosis has enlarged, this is reduced by using the J tip needle to take 2-3 interrupted sutures to reduce the diameter of the anastomosis to 25 mm. The suture is passed with the J tip needle and extracted with a retriever instrument on the other side and then it is taken out of the sheath (SH). An extracorporeal knot is tied and knot is pushed with the knot pusher into the stomach.

Transoral Gastric bypass (TOGB) is more challenging as it entails performing an anastomosis such as Gastrojejunostomy. However recently, endoscopic methods have been developed to perform bowel anastomosis. With the combination of these inventions, transoral gastric bypass with a rigid port is feasible.

### 5. Transoral Diagnostic Laparoscopy and Biopsy

A potential application of performing transoral pure NOTES is by using the USN 20.30 port (TOP-E) for placing a small 12 mm incision in the stomach just below the GE junction. The tunnel pusher sheath (SH) along with its blunt introducer (BI) and under 5 mm telescope is inserted through the gastric opening into the peritoneal cavity. Under vision inspection of the peritoneal cavity is done and biopsy is to be performed with one 5 mm instrument available. Even a flexible endoscope is passed from the transoral route from the tunnel pusher sheath (SH) into the peritoneal cavity for diagnostic peritoneoscopy and sos biopsy.

### 6. Transoral NOTES Cholecystectomy and Appendectomy

In this approach, TOP-E, a 2 cm incision is placed in the stomach just below the GE junction. A TOP-G is inserted under vision into the peritoneal cavity from this gastric opening and by using two other 5 mm angled instruments, cholecystectomy or appendectomy is performed. Finally the specimen is removed from the gastric incision and the transoral route. The gastric opening is closed by suturing or using the endostitch.

### 7. Transoral NOTES for Stomach Surgery

This includes procedures such as Transoral NOTES GJ. After TOP-G is inserted into the stomach, the posterior wall of stomach is opened to enter into the lesser sac. This avoids injury to the small bowel while opening anterior wall and directly entering the peritoneal cavity. A flexible endoscopy is then passed into the lesser sac. The peritoneum of the transverse mesocolon is opened close to the proximal jejunum. The endoscope is advanced into the peritoneal cavity. Thorough inspection of the peritoneal cavity is done for diagnostic peritoneoscopy. This proximal jejunal limb is withdrawn into the stomach opening. A posterior sutured gastrojejunostomy is constructed. The same method is used to perform anterior gastrojejunostomy.

### 8. Transoral NOTES Pancreatic Necrosectomy and Cystogastrostomy

is performed for large bulging pseudocyst into the stomach. Once the TOP-G port is inserted into the stomach, an incision is placed over the posterior gastric wall overlying the cyst. Once the pancreatic bed is entered, flexible endoscope is inserted into the pancreatic bed and necrosectomy is performed. Finally lavage is done and posterior cystogastrostomy is constructed with sutured or stapled technique.

### 9. Transoral NOTES Esophagectomy

Another potential application of using notes surgery for esophageal cancer is to perform transoral NOTES esophagectomy similar to transanal TME. Transoral Port (TOP) is used to divide the esophagus proximal to the tumour and come out into the mediastinum, just as is done in transanal TME for rectal cancer. Under vision, entire mediastinal esophagectomy and periesophageal lymphadenectomy is performed without disturbing the mediastinal pleura or the azygos vein. Neck incision is used to apply a SILS port for performing the mediastinal dissection of the esophagus. Finally once the peritoneal cavity is entered, TOP-E is converted to TOP-G to perform the steps in the abdomen such as gastric mobilization, left gastric vessels ligation. The specimen is removed from the transoral route or from the neck incision. Gastric pullup is performed and esophagogastric anastomosis is created with stapling or hand sewn.

The comparison with the prior art is mentioned herewith:

| | ThoracoLap. | NOTES |
|---|---|---|
| | Esophagectomy | Esophagectomy |
| | Currently Used | Just Innovated |
| Approach | Thoraco & Laparoscopic | NOTES - Transoral & |
| | | Umbilical |
| Invasion | Minimally invasive | Minimal invasion |
| Incisions | 4 Thoracic, 5 Abdominal | Neck One Neck and |
| | | One Umbilical |
| Scars | Multiple | Two |

Comparison with Prior Art

While the present Transoral port (TOP) of the present invention (P) and the novel methods for transoral endoscopic surgery of upper gastrointestinal tract using rigid platform (P) described herein above, following major comparative advantages of the present invention over the prior art such as diagnostic rigid esophagoscope, flexible endoscope, multiport laparoscopic surgery, SILS Surgery and transanal surgery with rigid port was established:
1. Rigid diagnostic Esophagoscopy: This was used only for diagnosis with primitive technology and in now outdated. The current invention has many therapeutic potentials and benefits which allows the current platform to perform advanced surgery.

| | Diagnostic Rigid | Operating |
|---|---|---|
| | Esophagoscope | Esophagoscope |
| | (Prior Art) | (New Innovation) |
| Design | Simple tube | Operating port |
| Use | Only diagnosis | Treatment |
| Optics | Simple | Hopkins Rod lens |
| | | Telescope |
| Light source | Cold light | Fibreoptic |
| Instrument | Single channel for | Atleast 3 ports for |
| | biopsy | telescope and two |
| | | instruments ports |
| Clinical | Outdated | Just Innovated |
| Relevance | | |

2. Laparoscopic Surgery: The multiport laparoscopic surgery is the current standard of care to perform these surgeries. However the advantages of the current invention over traditional laparoscopic surgery is summarised as below:

| | Multiport | Transoral |
|---|---|---|
| | Laparoscopic | endoscopic UGI |
| | surgery | surgery |
| Access | Abdominal | Oral |
| Approach | Multiport | Transoral |
| | Laparoscopy | endoscopic |
| Platform | Multiple rigid | Single TOP |
| | trocars | |
| Environment | Multiple instrument | Single incision |
| Incision, | Multiple small | None |
| Wounds | | |
| Scar | Multiple | None |
| Pain | + | None |
| Recovery | Fast | Faster |
| Complications | Less | Much less |

3. Single incision laparoscopic Surgery (SILS): Single incision upper gastrointestinal surgery has also been recently performed for various surgeries of the UGI tract. The salient differences between the SILS platform and the current invention are described below:

| | Single incision | |
|---|---|---|
| | Laparoscopic | Transoral UGI |
| | surgery(SILS) | Surgery |
| Access | Abdominal | Oral |
| Approach | Single incision | No incision |
| Platform | SILS | TOP |
| Incisionm | One-Umbilical | None |
| Wounds | | |
| Scar | One-hidden in | None |
| | umbilicus | |
| Pain | + | Painless |
| Recovery | Fast | Faster |
| Complications | Less | Much Less |

4. Flexible Endoscopy: Flexible endoscopy has been recently used for such advanced procedures. The major limitations of this access and the advantages of current innovation is summarised as below:

| | Flexible | |
|---|---|---|
| | endoscopy(Prior | Rigid |
| | Art) | Oesophagoscopy |
| Design | Flexible tube | Rigid platform |
| Use | Diagnostic & | Mainly Surgical |
| | Treatment | treatment |
| Anesthesia | Under LA/GA | Under GA |
| Personnel | Endoscopist | Surgeon |
| Instrument | One | Two |
| channel | | |
| Suturing | Not possible | Possible |
| Haemostasis | Limited | Better |

5. TransanalPlatform: Recently transanal endoscopic surgery has been started for performing rectal surgery with a rigid or a flexible port placed into the anal canal. The main differences with this approach and the current invention is mentioned below:

| | TransanalPlatform | |
|---|---|---|
| | (Prior Art) | Transoral platform |
| Use | Already in use | New Innovation |
| Application | Transanal surgery | Transoral surgery |
| | Rectal Surgery | Oesophageal |
| | | surgery |
| Diameter | 4 cm | 2 cm |
| Length | 4 cm to 25 cm | 30 cm to 45 cm |
| Difficulty | Difficult | Very difficult |

Thus the present invention has several advantages over the multiple prior arts as described here. It is a very promising and novel technique based on sound surgical principles of safety, efficacy and ease of use. Undoubtedly the techniques and the technologies needs to be used to put these inventions for the benefit of the mankind at large, to enable scarless and painless UGI surgery.

### Advantages of Present Invention

The Transoral port (TOP) of the present invention (P) and the novel methods for transoral endoscopic surgery of upper gastrointestinal tract using rigid platform (P) imparts following advantages over the prior arts; obviating its problems and challenges while surgery.
i. Present invention (P) eliminates the chances of injury by providing a blunt introducer (BI) that enables insertion of the present transoral port (TOP) into the transoral route of the patient without causing injury to its oropharyngeal area or to upper aerodigestive tract. Moreover, said blunt introducer (BI) is provided with under vision with a 5 mm to ensure the same.
ii. However, the present invention (P) obviates the problem of fixity by providing provision for suture fixation of the port with patients skin.
iii. The present invention (P) minimizes the instrument clashing by providing specially designed curved instruments and related techniques.
iv. Present invention (P) uses continuous flow CO2 insufflator to circumvent the issue of air leakage.
v. A suction port (SP) is provided in present invention (P) to keep sucking of the smoke that is produced.
vi. Present invention (P) provides bevel at the end of the transoral ports facilitating the surgical procedures in the wall of the esophagus.
vii. Present invention (P) provides a tunnel pusher tube to develop a sub-mucosal tunnel, and to perform NOTES surgery.

1. The present novel equipment for transoral endoscopic surgery of upper gastrointestinal tract using rigid platform (P) mainly includes the Transoral Port (TOP) which is used for performing natural orifice scarless surgery of the upper gastrointestinal tract;
wherein, said Transoral port (TOP) mainly comprises of following parts:
Sheath (SH),
Blunt Introducer (BI),
Telescope (TE),
Diaphragm (DI),
Silicon washers (SW),
Tunnel pusher tube (TP),
Air port (AP),
Suction port (SP),
Bevel (AB or PB),
Markings (MA),
Monitor (MO);
wherein further:
said Sheath (SH) is a hollow tube; which is inserted from access route or the natural orifice; including transoral route and it remains in the patient through which the instruments pass into the body to perform surgery; said sheath (SH) is introduced with the help of blunt introducer (BI) which has an incorporated 5 mm channel to introduce telescope and insert the Transoral port (TOP) under vision; Said sheath (SH) has insufflations and suction ports (SP) which are also used as fixation device;
Said Diaphragm (DI) is screwed air tight to the sheath (SH) on which a silicon washer is applied;
Said Silicon Washers (SW) fits onto said diaphragm (DI) have a plurality of openings with airtight seal to insert multiple long and narrow (laparoscopic) instruments (IN1, IN2) through it into the sheath (SH); Said airtight seal prevents gas leakage; Said washer (SW) can be changed for different steps of the surgery depending on the nature of instrument to be used; Different embodiments for present Transoral Port (TOP) uses washers (SW) with different number of openings; which include:
i. A washer (SW) for 20 mm sheath (SH) has three openings of 5 mm diameter each in a first embodiment of the present invention (P); said three openings are utilized to insert into the sheath (SH), while doing the surgery, a 5 mm telescope and two 5 mm instruments (IN1, IN2);
ii. A washer (SW) for 20 mm sheath (SH) has two openings of 10 mm and 5 mm diameter each in a second embodiment of the present invention (P); Said washer (SW) can be designated as 10,5 washer (SW) wherein a 5 mm telescope and one 10 mm instrument are inserted through the openings;
iii. A washer (SW) for 20 mm sheath (SH) has one opening of 12 mm diameter each in a third embodiment of the present invention (P); Said washer (SW) can be designated as 12 washer (SW); Said 12 mm washer is used to pass tunnel pusher tube;
Said Telescope (TE), in a preferred embodiment is a high definition (HD) telescope to provide a clear image, preferably it is a 5 mm telescope of around 50 cm (varying from 30 cm to 60 cm) length with video camera attached on it; and is used to provide the vision for surgery; On the telescope, a fibre-optic light cable is attached for providing light for the surgery; The telescope is generally held by the assistant surgeon;
Said Air port (AP) is used for CO₂ insufflation at a flow of 10-20 L/min with a pressure of 15 mm Hg (varying from 10-20 mm Hg); this distends the stomach and creates a space for surgery;
Said Suction port (SP) is used for suction of smoke while using the electrocautery during the surgical procedures;
Said Instruments (IN1, IN2) have a plurality of long, preferably 35 cm to 50 cm depending on sheath (SH) used (varying from 20 cm to 60 cm), and thin, preferably 2 to 5 mm (varying from 1 mm to 6 mm) instruments are used in different configurations for performing the surgical procedure through present rigid platform; Said instruments include maryland dissector, atraumatic graspers, hook electrode, star electrode, needle holder, suction, bipolar cautery; the present invention (P) also embodies special instruments that are developed having a curved tip for working in present transoral port with small diameter; they also have curves on the proximal end to avoid instrument fighting; These instruments are either 3 or 5 mm and have an extra length of 50 cm; Said special instruments include:
Star Electrode designed for creating submucosal space and performing myotomy in TOEM; Said star electrode is a three point star electrode with an active tip;
J tip needle along with thread retriever is developed to facilitate suturing of horizontal defects in a small space;
Said Sheath (SH) has bevel (AB or PB) at its distal end which is a cut out to perform surgery;
Said Sheath (SH) of the Transoral port for Esophagus (TOP-E) and Stomach (TOP-G) have markings on their anterior surface for visibility and orientations for the surgeon, as to the surface of the organ, whether he or she is anterior or posterior and as to the position of the TOP to the length of esophagus or stomach.

2. The present novel equipment for transoral endoscopic surgery of upper gastrointestinal tract using rigid platform (P) as claimed in claim 1; wherein present Transoral Ports (TOP), based on its applicability includes:
i. Transoral Port for surgery of the Esophagus (TOP-E) which is having a working length of 30 cm and is specially designed for performing cardiomyotomy for achalasia cardia (TOEM) and transoral Esophagectomy;
ii. Transoral port for surgery of the GE junction and Stomach (TOP-G) which is having a working length of 45 cm, which is specially designed to perform Transoral Fundoplication (TOF), Transoral bariatric surgery (TOBS) and NOTES UGI surgery.

3. The present novel equipment for transoral endoscopic surgery of upper gastrointestinal tract using rigid platform (P) as claimed in claim 1; wherein said transoral ports (TOP) have a 20 mm internal working diameter.

4. The present novel equipment for transoral endoscopic surgery of upper gastrointestinal tract using rigid platform (P) as claimed in claim 1; wherein said transoral port (TOP) is rigid and is made of Stainless Steel or Titanium; which is thus light in weight, easily autoclavable and biocompatible.

5. The present novel equipment for transoral endoscopic surgery of upper gastrointestinal tract using rigid platform (P) as claimed in claim 1; wherein said sheath (SH) is of different lengths for esophageal and gastric applications as under:
i. Sheath (SH) for transoral port of esophagus (TOP-E) with diameter of 20 mm (from 15 to 25 mm), and a working length of 30 cm (from 25 to 40 cm) for Esophageal surgery; Said sheath (SH) passes from the mouth through the upper esophageal (cricopharyngeal sphincter) into the esophagus under direct vision with the help of blunt introducer (BI);
ii. Sheath (SH) for Transoral surgery for GE junction & Gastric surgery (TOP-G) such as Fundoplication and bariatric surgeries has a length of 45 cm (varying from 40 to 60 cm with a diameter of 2 cm; Said sheath (SH) passes from the mouth through the upper oesophageal (cricopharymgeal sphincter) into the stomach under direct vision with the help of blunt introducer (BI).

6. The present novel equipment for transoral endoscopic surgery of upper gastrointestinal tract using rigid platform (P) as claimed in claim 1; wherein said sheath (SH) is made of stainless steel.

7. The present novel equipment for transoral endoscopic surgery of upper gastrointestinal tract using rigid platform (P) as claimed in claim 1; wherein said Diaphragm (DI) made from biocompatible Teflon and Stainless Steel so as to not to make the entire port very heavy.

8. The present novel equipment for transoral endoscopic surgery of upper gastrointestinal tract using rigid platform (P) as claimed in claim 1; wherein said silicon washer (SW) are made of biocompatible Silicon.

9. The present novel equipment for transoral endoscopic surgery of upper gastrointestinal tract using rigid platform (P) as claimed in claim 1; wherein said bevel (AB or PB) on said sheath (SH) of present transoral port (TOP) includes:
i. Bevel (PB) on said sheath (SH) of the Transoral Port (TOP) for Esophagus (TOP-E) is at its distal end which is a cut out to engage the esophagus posteriorly for performing posterior cardiomyotomy; said bevel is for 10 mm depth (varying from 5 to 20 mm) and occupying 50% circumference (ranging from 25-75% circumference);
ii. Bevel (AB) on said Sheath (SH) of the Transoral port for GE junction and Gastric Surgery (TOP-G) is anteriorly on its distal end where fundoplication needs to be performed; said bevel is anteriorly to perform an anterior Dorr fundoplication.

10. The present novel equipment for transoral endoscopic surgery of upper gastrointestinal tract using rigid platform (P) as claimed in claim 1; wherein another embodiment of the present invention (P) provides a Tunnel Pusher sheath (TP); which is made of Stainless Steel (SS) and is 12 mm diameter (varying from 8 to 16 mm) and has a length of 45 cm (varying from 40 to 60 cm); It has two ports at proximal end for insufflation and smoke evacuation; The tunnel pusher sheath (SH) is also inserted under vision with a blunt introducer (BI); said Tunnel Pusher sheath (TP) uses a silicon washer (SW) which is a washer (SW) with two openings with 5 mm diameter each to accommodate one 5 mm telescope and another 3 or 5 mm instrument.

11. Present trans-oral equipments and methods for transoral endoscopic surgery of upper gastrointestinal tract using rigid platform (P) as claimed in claim 1; wherein present Transoral Port (TOP) is used to perform the Trans-oral Endoscopic myotomy (TOEM) for Achalasia Cardia; wherein said surgery of cardiomyotomy with transoral approach;
Said novel method to perform said Transoral Endoscopic myotomy (TOEM) uses present novel equipments (P) with following steps:
i. Once the Transoral Port (TOP) is inserted under vision, said Blunt Introducer (BI) is removed, diaphragm (DI) is applied and the platform is fixed to the patient with two sutures; said sheath (SH) is fixed in such a way that the bevel (PB) is posterior and the markings (MA) are anterior; then a diagnostic flexible endoscopy is then performed through the transoral port to assess the distance of the GE junction and plan the myotomy and the tunnel length; Insufflation with CO₂ is started and ETCO2 monitoring is done by the anesthetist to ensure that hypercarbia does not occur; this follows Painting of local oesophageal mucosa is done with dilute Povidone Iodine and Flushing with Saline is done;
ii. The procedure of endoscopic cardiomyotomy is now started wherein a 5 mm Telescope (TE) 30 degree angled, 50 cm long telescope is chosen to perform the myotomy; this telescope (TE) is used with standard laparoscopic instruments to perform the myotomy; Alternatively a standard 5 mm 30 degree telescope (TE) is used with long bariatric length instruments to perform the myotomy; Using differential length of instruments and telescope (TE) is to avoid instrument and telescope (TE) clashing in a very small space of 2 cm diameter of present transoral port (TOP);
iii. the first step in this surgery is to create a submucosal space in the esophagus; then Injection of dilute 1:100 methylene blue is done by an aspiration needle in the submucosal space to create a mucosal bulge; this lifts the mucosa and allow a submucosal tunnel to be created; 10-20 cc of dilute methylene blue is injected and mucosal bulging is observed;
iv. a Mucosal incision is placed starting at 6' o clock position on the esophagus to perform a posterior myotomy; a longitudinal incision is preferred so that mucosal closure with suturing becomes easier in view of the horizontal suture line, which is then closed with the J tip needle of the special instruments (IN1 or IN2) of the present invention (P); Alternatively a vertical mucosal incision is used and closed with standard suturing done with needle holder or endostitch (IN1, IN2); another alternative is to do an anterior myotomy which has shown to have equivalent results as posterior myotomy;
v. the next step is to create sub-mucosal tunnel is the next goal; Gentle dissection by a maryland dissector creates a space in the submucosal area for accommodating a balloon; A CRE balloon from 10-12 mm is used to create a submucosal space so that the submucosal pusher tube is inserted into the newly created submucosal space;
vi. Further, a Tunnel pusher (TP) Tube of 12 mm diameter and a 5,5 silicon washer (SW) along with blunt under vision introducer (BI) with CO2 insufflation is inserted into the submucosal space that has been created; Insufflation with CO₂ is started to enlarge this space; Under vision of a 5 mm 0 degree telescope and a hook or a triangular star instrument which is slightly curved at the tip, the submucosal space is developed distally into the esophagus; a long 12-15 cm of submucosal tunnel is created, comprising approx.; 10-12 cm in the esophagus and around 2-3 cm into the stomach; the tunnel is elongated by going beyond GE junction into stomach for 2-3 cm; The crisscross fibres of the GE junction is apparent and signifies the presence of GE junction; Small submucosal blood vessels are carefully coagulated and divided to prevent bleeding wherein if bleeding is encountered, control is achieved with bipolar coagulation forceps; Care is taken to do each step under direct vision and avoiding mucosal perforation;
vii. Once the tunnel is complete, the Myotomy is started—For myotomy, a tunnel pusher (TP) tube of 45 cm length with a posterior bevel (PB) is used to perform a posterior myotomy; A 5 mm 30 degree angled telescope (TE) is preferred to perform the myotomy; The myotomy is restricted to the circular fibres or alternatively a full thickness myotomy is performed; The recommended length of myotomy is approx.; 10-12 cm out of which, approx 8-10 cm is in esophagus and 2-3 cm into stomach; The myotomy is started about 2-3 cm; distal to the mucosal opening into the esophagus to create a flap like effect upon closing the mucosa; Once the myotomy is complete beyond the GE junction, haemostasis is achieved; The myotomy is performed by a ⅗ mm hook electrode or a star electrode with a cutting or a blended current; It is advisable that all steps are done under vision; One special problem of smoke evacuation needs to be addressed due to the narrow diameter of the platform; Several options to circumvent this problem is by using a hook with inbuilt suction port (SP), simultaneous cautery and suction machine, foot controlled suction, dual suction etc; To evacuate the smoke, the telescope may need to be withdrawn repeatedly while cautery is being used;
viii. The mucosal closure is performed with sutures or endostitch; For a suture closure of horizontal incision, said special instrument (IN1/IN2) i.e. J tip needle device is used to pass the needle with thread which is then retrieved with a special retriever instrument (IN1/IN2) which are 2 mm in diameter; Alternatively, for closing a vertical incision, a vicryl 3/0 is used on a 27 mm ⅝^{th} circle needle; The needle is grasped by the needle holder and taken into the esophagus from the Transoral Port (TOP); The stitch is taken without dislodging the needle; The needle is grasped and extracted from the Transoral Port (TOP); An extracorporeal knot is preferred and tightened; Another simple or figure of 8 suture is taken and this completes the mucosal closure; Generally 2-3 sutures are necessary to close the mucosal incision; For endostitch closure, a new Silicon washer 10,5 is needed to be applied on the Diaphragm (DI) of the Transoral Port (TOP); A 10 mm endostitch is passed into the esophagus with an angled 5 mm telescope of 30 degree; Endostitch is taken on both edges of the mucosa and endostitch is removed and an extracorporeal knot is tied and pushed into the esophagus with a knot pusher (KP).

12. Present novel equipments and methods for transoral endoscopic surgery of upper gastrointestinal tract using rigid platform (P) as claimed in claim 1; wherein it is used for Transoral endoscopic Esophagectomy (TOE) or Hybrid NOTES Esophagectomy (HNE);
in the said surgery, present Transoral Port (TOP) is used to divide the esophagus proximal to the tumour and come out into the mediastinum, just as is done in transanal TME for rectal cancer; Under vision, entire mediastinal esophagectomy and periesophageal lymphadenectomy is performed without disturbing the mediastinal pleura or the azygos vein;
said TOP-E is inserted into the esophagus just proximal to the tumour and fixed in position and Insufflation of CO2 through said Air Port (AP) is started; where after a purse string suture is taken into the port and the esophagus is closed 1-2 cm proximal to the tumour; the knot is again tied extracorporeally and pushed into the esophagus;
an incision is placed encircling the esophagus and is deepened through the esophagus to enter into the mediastinum; the incision is extended all around in the esophagus; care is taken not to injure mediastinal structures including trachea, bronchus and azygos veins; With the help of blunt grasper in one hand and hook or harmonic scalpel in another help the entire esophagus is completely separated from adjoining structures; Haemostasis is achieved; Peritoneum is entered and then SILS umbilical approach is used to complete the abdominal part of the procedure; Also complete NOTES esophagectomy is achieved by inserting the TOP-G port into the abdomen after peritoneal entry and performing the abdominal steps from the transoral route.

13. Present novel equipments and methods for transoral endoscopic surgery of upper gastrointestinal tract using rigid platform (P) as claimed in claim 1; wherein Transoral Port (TOP-G) is used to do Transoral Endoscopic Fundoplication (TOF); for which, said Transoral Port (TOP-G) is inserted from the mouth into the esophagus and then a partial anterior Don Fundoplication is done by hitching the fundus of the stomach to the GE junction with three interrupted sutures to create an angulation of the GE junction and create an antireflux barrier to prevent gastro oesophageal reflux; this surgery is also done in conjunction with a cardiomyotomy to prevent reflux commonly seen after cardiomyotomy;
alongwith TOEM, Said Transoral Endoscopic Fundoplication (TOF) is performed before doing a myotomy preferably, as it does not compromise the myotomy or the oesophageal mucosal closure while placing the transoral ports (TOP); alternatively it is done after the myotomy;
the steps for Transoral Fundoplication (TOF) either for hiatus hernia or for achalasia cardia is the same and is described below:
Sheath (SH) of Transoral Port (TOP) for Gastric surgery (TOP-G) of 45 cm length, with 2 cm diameter is inserted under vision of a 5 mm telescope (TE) into the esophagus;
wherein said sheath (SH) has an anterior bevel to perform anterior Don fundoplication;
the silicon washer (SW) of second embodiment of the present invention with two openings—10 mm and 5 mm is attached to Diaphragm (DI) for suturing with endostitch 10 mm; the suturing of the fundus with the GE junction is performed with assistance of any of the following techniques:
i. Endoscopic guidance—a Screw like instrument (IN1/IN2) is inserted from the instrument channel of a flexible 10 mm endoscope (TE) to hook the fundus of stomach after retroflexion and pull the fundus; said endoscope (TE) is then taken out keeping the instrument channel in position; Endostitch is then inserted from the 10 mm port, suturing of the fundus with the anterior part of GE junction performed; extracorporeal knotting is preferred over intracorporeal knotting; wherein three such sutures are placed at 9, 12, 3 o clock position (ST1, ST2, ST3);
ii. Laparoscopic guidance—A 5 mm telescope (TE) at umbilicus gives a vision of the GE junction with the help of Hammock retractor retracting the left lobe of the liver; wherein a 3 mm trocar is placed just underneath the xiphoid process and a 3 mm blunt instrument depresses anterior fundus of the stomach; Under this vision, transoral suture is passed by the transoral surgeon; wherein this approach needs a two team approach and two endovision systems simultaneously to perform the fundoplication; Similarly three sutures are taken with laparosopic assistance and all are tied; Alternatively from a SILS port at the umbilicus and three 5 mm ports and a Hammock retractor in position to retract the left lobe of the liver, an anterior Don fundoplication is performed from a completely laparoscopic U NOTES approach;
iii. Flexible paediatric endoscopic guidance—A 5 mm, 100 cm long flexible paediatric endoscope is used to assist taking fundal sutures by using a rat tooth grasper from the instrument channel of 2.2 mm to pull the fundus and take the sutures hitching it to the GE junction;
iv. J Needle—said J needle (IN1/IN2) is also be used to perform the fundoplication with along port with the help of the thread retriever.

14. Present novel equipments and methods for transoral endoscopic surgery of upper gastrointestinal tract using rigid platform (P) as claimed in claim 1; wherein Transoral Endoscopic Bariatric surgery (TOBS) procedures include Transoral endoscopic gastric sleeve—TOGS, revisional procedures for weight gain after gastric bypass surgery such as Transoral revision after bariatric surgery—TORBS and transoral gastric bypass (TOGB); wherein said procedures involve following steps:
The first step in Transoral gastric Sleeve (TOGS) is introduction of TOP-G into the stomach followed by plicate the greater curvature of the stomach with multiple sutures to reduce the capacity of the stomach; this is done at several levels of the stomach starting from distal stomach and gradually progressing towards the proximal stomach; the knots are tied extracorporeally and pushed back into the stomach; at the end of the procedure the capacity of the stomach is reduced; this is done with plication sutures or even stapling guns currently available introduced from the transoral approach;
For revision of laparoscopic gastric bypass, when the anastomosis has enlarged, this is reduced by using the J tip needle (IN1, IN2) to take 2-3 interrupted sutures to reduce the diameter of the anastomosis to 25 mm; then the suture is passed with the J tip needle (IN1, IN2) and extracted with a retriever instrument on the other side and then it is taken out of the sheath (SH); an extracorporeal knot is tied and and knot is pushed with the knot pusher into the stomach;
Transoral Gastric bypass (TOGB) entails performing an anastomosis such as Gastrojejunostomy and transoral gastric bypass with a rigid port is also feasible using present novel equipments and methods for transoral endoscopic surgery of upper gastrointestinal tract using rigid platform (P).

15. Present novel equipments and methods for transoral endoscopic surgery of upper gastrointestinal tract using rigid platform (P) as claimed in claim 1; wherein Transoral diagnostic laparoscopy and biopsy using TOP-E wherein further a small 12 mm incision is placed in the stomach just below the GE junction; then the tunnel pusher sheath (SH) along with its blunt introducer (BI) and under 5 mm telescope (TE) is inserted through the gastric opening into the peritoneal cavity; after which Under vision inspection of the peritoneal cavity is done and biopsy is to be performed with one 5 mm instrument (IN1/IN2); alternatively, a flexible endoscope is passed from the transoral route from the tunnel pusher sheath (SH) into the peritoneal cavity for diagnostic peritoneoscopy and sos biopsy.

16. Present novel equipments and methods for transoral endoscopic surgery of upper gastrointestinal tract using rigid platform (P) as claimed in claim 1; wherein Transoral NOTES Cholecystectomy and appendectomy wherein said TOP-E is used, a 2 cm incision is placed in the stomach just below the GE junction and said TOP-G is inserted under vision into the peritoneal cavity from this gastric opening and by using two other 5 mm angled instruments, cholecystectomy or appendectomy is performed; finally the specimen is removed from the gastric incision and the transoral route; the gastric opening is closed by suturing or using the endostitch.

17. Present novel equipments and methods for transoral endoscopic surgery of upper gastrointestinal tract using rigid platform (P) as claimed in claim 1; wherein Transoral NOTES for stomach surgery and gastrojejunostomy which includes procedures such as Transoral NOTES GJ is performed; wherein After TOP-G is inserted into the stomach, the posterior wall of stomach is opened to enter into the lesser sac; A flexible endoscope is then passed into the lesser sac; The peritoneum of the transverse mesocolon is opened close to the proximal jejunum and the endoscope is advanced into the peritoneal cavity; thorough inspection of the peritoneal cavity is done for diagnostic peritoneoscopy; this proximal jejunal limb is withdrawn into the stomach opening; a posterior sutured gastrojejunostomy is constructed.

18. Present novel equipments and methods for transoral endoscopic surgery of upper gastrointestinal tract using rigid platform (P) as claimed in claim 1; wherein Transoral NOTES pancreatic necrosectomy and cystogastrostomy is performed for large bulging pseudocyst into the stomach wherein, once the TOP-G port is inserted into the stomach, an incision is placed over the posterior gastric wall overlying the cyst; Once the pancreatic bed is entered, flexible endoscope is inserted into the pancreatic bed and necrosectomy is performed; finally lavage is done and posterior cystogastrostomy is constructed with sutured or stapled technique.

19. Present novel equipments and methods for transoral endoscopic surgery of upper gastrointestinal tract using rigid platform (P) as claimed in claim 1; wherein surgery for esophageal cancer is performed using present Transoral Port (TOP) to divide the esophagus proximal to the tumour and come out into the mediastinum; Under vision, entire mediastinal esophagectomy and periesophageal lymphadenectomy is performed without disturbing the mediastinal pleura or the azygos vein; then neck incision is used to apply a SILS port for performing the mediastinal dissection of the esophagus; Finally once the peritoneal cavity is entered, TOP-E is converted to TOP-G to perform the steps in the abdomen such as gastric mobilization, left gastric vessels ligation; The specimen is removed from the transoral route or from the neck incision; Gastric pullup is performed and esaphagogastric anastomosis is created with stapling or hand sewn.
